(19) United States
(12) Patent Application Publication
(10) Pub. No.: US 2024/0389633 A1
(43) Pub. Date: Nov. 28, 2024

(54) METHOD FOR IMPROVING SENSORY QUALITY OF CUT ROLLED STEM USING GANODERMA LUCIDUM FERMENTATION BROTH

(71) Applicant: CHINA TOBACCO SICHUAN INDUSTRIAL CO., LTD., Chengdu (CN)

(72) Inventors: Minchang LIU, Chengdu (CN); Wu WEN, Chengdu (CN); Yaping MA, Chengdu (CN); Rongya ZHANG, Chengdu (CN); Tingjie BAO, Chengdu (CN); Ruoyu WEN, Chengdu (CN); Ruifang GU, Chengdu (CN); Lingying HE, Chengdu (CN); Zhengrong ZENG, Chengdu (CN); Yang LIU, Chengdu (CN); Xianyong AN, Chengdu (CN); Chunxiao LUO, Chengdu (CN)

(73) Assignee: CHINA TOBACCO SICHUAN INDUSTRIAL CO., LTD., Chengdu (CN)

(21) Appl. No.: 18/661,756

(22) Filed: May 13, 2024

(30) Foreign Application Priority Data
May 26, 2023 (CN) 202310610698.8
May 26, 2023 (CN) 202310611250.8

(51) Int. Cl. A24B 5/16; A24B 3/04; A24B 15/20; C12N 1/14; C12R 1/645

(52) U.S. Cl. CPC A24B 5/16; A24B 3/04; A24B 15/20; C12N 1/145; C12R 2001/645

(57) ABSTRACT

A method for improving a sensory quality of a cut rolled stem using a Ganoderma lucidum fermentation broth is provided, including: subjecting Ganoderma lucidum to first-stage cultivation and second-stage expanded cultivation to obtain an extracellular enzyme-containing fungal solution, and filtering the extracellular enzyme-containing fungal solution with a plate and frame filter to obtain a clear fermentation broth without obvious solids; applying the fermentation broth to a tobacco stem or a cut rolled stem, and allowing solid-state fermentation to obtain a fermented tobacco stem or cut rolled stem; and subjecting the fermented tobacco stem or cut rolled stem to expansion, inactivation, and excess water removal. After the fermentation, cell wall substances such as lignin and cellulose are degraded and utilized, which reduces the occurrence of undesirable smells such as a woody smell and a pungent odor, and improves a sensory quality of the cut rolled stem.

### CROSS REFERENCE TO THE RELATED APPLICATIONS

This application is based upon and claims priority to Chinese Patent Application No. 202310610698.8, filed on May 26, 2023, and Chinese Patent Application No. 202310611250.8, filed on May 26, 2023, the entire contents of which are incorporated herein by reference.

### TECHNICAL FIELD

The present disclosure relates to the technical field of tobacco processing, and in particular to a method for improving a sensory quality of a cut rolled stem using a Ganoderma lucidum fermentation broth.

### BACKGROUND

Tobacco stems are a natural constituent part of tobacco leaves, and tobacco stems are tobacco stalks. The weight of the tobacco stem is about 25% to 30% of the weight of the tobacco leaf. The tobacco stems can be made into cut rolled stems through sieving, stem washing and soaking, heating and humidifying, stem storage, shredding, and fragrance addition. Cut rolled stems processed from tobacco stems can be used in cigarettes and have the advantages of excellent filling performance, adjusting the smoke state, reducing the tar content, etc. However, the cut rolled stems also have sensory defects such as a woody smell, irritation, and burning sensation, and have a low utilization rate, which is not conducive to the comprehensive utilization of tobacco resources. According to chemical composition analysis, the tobacco stems have high contents of cell wall substances such as lignin, cellulose, hemicellulose, and pectin, which are the main cause of sensory defects such as a heavy offensive woody odor, strong irritation, and a heavy burning sensation of cut rolled stems.

In recent years, with the popularization of the concept of environmental protection, research efforts on the improvement of the smoking quality of cut rolled stems and the utilization rate of tobacco stems have been increased in the tobacco industry. Currently, biodegradation methods for cell wall substances in tobacco stems mainly include an enzymatic preparation degradation method and a bacterial preparation degradation method. However, the enzymatic preparation has a relatively simple effect, and the bacterial preparation has a relatively low degradation efficiency. With regard to the use of Ganoderma lucidum in cigarettes, the patent CN108142988B discloses a preparation method of a Siraitia grosvenorii extract for fragrance-enhancing and moisturizing, and the patent CN103965369B discloses a Ganoderma lucidum polysaccharide and an extraction and purification method thereof to improve a moisturizing effect for tobacco. In all of the above techniques, Ganoderma lucidum is used in the moisturizing for tobacco. There has been no report on a method for reducing the lignin content of tobacco using a Ganoderma lucidum fermentation broth to improve the sensory quality of a cut rolled stem.

### SUMMARY

An objective of the present disclosure is to provide a method for improving a sensory quality of a cut rolled stem using a Ganoderma lucidum fermentation broth to solve the above problems. After a cut rolled stem is treated by the method of the present disclosure, cell wall substances such as lignin in the cut rolled stem are well degraded, and a sensory quality of the cut rolled stem is improved significantly. The method of the present is easy to implement and the quality of the obtained cut rolled stem is stable.

The present disclosure adopts the following technical solutions to allow the above objective:

A method for improving a sensory quality of a cut rolled stem using a Ganoderma lucidum fermentation broth is provided, including the following steps:
step 1, fermentation broth preparation: subjecting Ganoderma lucidum to first-stage cultivation and then to second-stage expanded cultivation in a fermentation tank to obtain a fungal solution including extracellular enzymes such as laccase, cellulase, and pectinase, and filtering the fungal solution to obtain a clear fermentation broth;
step 2, conducting a pretreatment on a tobacco stem to obtain a pretreated tobacco stem or a cut rolled stem, where the pretreatment is one or more treatments of sieving, stem washing and soaking, heating and humidifying, stem storage, and shredding;
step 3, fermentation broth application: applying the fermentation broth to the pretreated tobacco stem or the cut rolled stem;
step 4, solid-state fermentation: subjecting the pretreated tobacco stem or the cut rolled stem to the solid-state fermentation to obtain a fermented tobacco stem or a fermented cut rolled stem; and
step 5, drying the fermented tobacco stem or the fermented cut rolled stem.

In the step 1, a medium for the first-stage cultivation and the second-stage expanded cultivation is a potato dextrose broth (PDB) medium or a potato dextrose agar (PDA) medium; and with the laccase in the fungal solution as a representative protease, an enzymatic activity of the laccase at the end of cultivation is 50,000 U/L to 300,000 U/L.

Optionally, in the step 1: (1) for the first-stage cultivation, the Ganoderma lucidum is inoculated at an inoculum size of 1% to 5%, and is cultivated for 4 d to 6 d at a temperature of 25° C. to 35° C. and a rotational speed of 80 rpm to 120 rpm; (2) the cultivation tank needs to undergo space sterilization and filled tank sterilization before the second-stage expanded cultivation, and the second-stage expanded cultivation is conducted for 4 d to 5 d with pressure difference inoculation as an inoculation mode to prevent impurity bacterial infection, an inoculum size of 5% to 10%, a ventilation ratio of 0.4 to 0.8, a rotational speed of 80 rpm to 120 rpm, and a temperature of 25° C. to 35° C. to obtain the fungal solution including extracellular enzymes such as laccase, cellulase, and pectinase.

In the step 1, the fungal solution is filtered with a plate and frame filter, and the plate and frame filter has a mesh diameter of smaller than or equal to 1,000 μm, such as 500 μm and 1,000 μm. After the filtering, solids such as mycelia in the fungal solution are filtered out to obtain the clear fermentation broth without obvious solids. The fermentation broth obtained is stored at a temperature of lower than or equal to 4° C. for later use to prevent the fermentation broth from losing its activity early.

In the step 4, the solid-state fermentation is conducted for 4 h to 24 h at a fermentation temperature of 30° C. to 60° C. and a relative humidity of 70% to 90%.

When different processes are adopted for the pretreatment, different solutions are adopted for the subsequent fermentation broth application and solid-state fermentation:

### A First Solution

In the step 2, the pretreatment includes the sieving, the stem washing and soaking, the heating and humidifying, the stem storage, and the shredding, sequentially.

The sieving is conducted with a sieve pore size of 2 mm to 4 mm for removing fine stems and a sieve pore size of 8 mm to 12 mm for removing stem ends. The sieving is conducted to remove stem ends and fine stems.

The stem washing and soaking is as follows: washing a tobacco stem in a stem washing and soaking device with a circulating water temperature of 20° C. to 60° C. to remove impurities such as dust, sand, and metals, during which the tobacco stem is both washed and soaked.

The heating and humidifying is as follows: storing a tobacco stem in a heating and humidifying device with a steam pressure of 0.1 Mpa to 0.5 Mpa for 2 h to 48 h.

The stem storage is as follows: storing the tobacco stem obtained after the heating and humidifying.

The shredding is as follows: cutting a tobacco stem by a double-cutting process to obtain a cut rolled stem, where a thickness of the stem sheet obtained by the first cut is 0.1 mm to 0.3 mm and a width of the cut rolled stem obtain by the second cut (namely, re-cutting) is 0.8 mm to 1.2 mm.

After the pretreatment, a cut rolled stem with a moisture content of 25% to 35% is obtained. Before the fermentation broth is applied, the cut rolled stem is heated and humidified to make the cut rolled stem loose and thus improve the uniformity of application of the fermentation broth to the cut rolled stem. The fermentation broth is applied as follows: spraying a diluted fermentation broth on a surface of the cut rolled stem by a feeding machine with an application proportion of 0.5 wt % to 2.0 wt %. In addition to the application of the fermentation broth, the feeding machine may apply a sugar material, water, or the like to the cut rolled stem. In this case, the feeding machine has a hot air temperature of 40° C. to 50° C., a water application proportion of 0% to 3%, and a sugar material application proportion of 1.0% to 3.0%, where hot air is heated by a heat exchanger, the fermentation broth, the water, and the sugar material are injected with compressed air, and the direct steam injection is avoided throughout this process to prevent the early inactivation of the fermentation broth.

The fermentation broth is diluted with water and then sprayed on the surface of the cut rolled stem. After the fermentation broth is diluted, an enzymatic activity of laccase is 10,000 U/L to 50,000 U/L.

The solid-state fermentation is conducted in a cut rolled stem storage cabinet of a leaf storage room, where the leaf storage room has a temperature of 30° C. to 40° C. and a relative humidity of 70% to 90%, and a storage time is 4 h to 24 h. During this process, the fermentation broth fully penetrates into the cut rolled stem in the high-temperature and high-humidity environment, and cell wall substances in the cut rolled stem are degraded into small-molecule substances such as polysaccharides under the action of laccase, cellulase, pectinase, or the like in the fermentation broth.

In the step 5, the cut rolled stem is expanded and inactivated with a high-temperature steam before being dried, and then dried by a roller dryer to remove excess water. For the expansion of the cut rolled stem, a steam pressure is 0.3 MPa to 0.5 MPa, a steam flow rate is 600 kg/h to 900 kg/h, and a temperature of the cut rolled stem is 80° C. to 95° C., such that the proteases in the fermentation broth in the cut rolled stem are inactivated under the action of the high-temperature steam to terminate the fermentation process. The roller dryer has a wall temperature of 110° C. to 140° C. and a hot air temperature of 100° C. to 120° C., and the drying is conducted to remove the excess water in the cut rolled stem. A cut rolled stem product discharged at an outlet of the roller dryer has a moisture content of 12.5% to 13.5%.

After the drying, impurities such as stem lumps and wet masses in the cut rolled stem are removed through winnowing, and finally, fragrances are added to obtain a finished cut rolled stem. The winnowing is conducted with a wind speed of 7 m/s to 15 m/s to remove impurities such as stem slivers, stem lumps, and wet masses in the cut rolled stem. After the winnowing, a sieving procedure is adopted to remove fragments and debris in the cut rolled stem. After the fragrances are evenly applied, a resulting cut rolled stem product is stored in a cut rolled stem storage cabinet to finally obtain a finished cut rolled stem with a moisture content of 11.5% to 12.5%.

In the first solution, the fermentation broth is applied to a surface of a cut rolled stem by the feeding machine during the stage of applying the sugar material to the cut rolled stem to make the cut rolled stem fully and evenly contact the fermentation broth, and subsequently, a fermentation process is terminated through expansion, drying, and high-temperature inactivation to guarantee the consistency of fermentation qualities of different batches.

### A Second Solution

In the step 2, the pretreatment includes the sieving, and after the pretreatment, the pretreated tobacco stem is obtained. The fermentation broth is applied as follows: adding the fermentation broth to circulating water in a stem washing and soaking device, and with an enzymatic activity of laccase in the circulating water in the stem washing and soaking device controlled at 3,000 U/L to 10,000 U/L and a temperature of the circulating water controlled at 40° C. to 60° C., washing and soaking the pretreated tobacco stem for 20 s to 120 s to obtain a washed and soaked tobacco stem with a moisture content of 20% to 30%, where after the tobacco stem is washed and soaked, the fermentation broth is absorbed, and impurities are washed away. The larger the amount of the fermentation broth added, the more fully the cell wall substances in a tobacco stem are degraded, but there will be an inharmonious smell similar to feather scorching. The higher the circulating water temperature and the longer the washing and soaking time, the higher the moisture content of a tobacco stem, which is conducive to the improvement of absorption and penetration efficiencies of the fermentation broth, but increases a power required for the subsequent expansion and drying and leads to a specified scorching smell. The fermentation broth is directly applied during the stem washing and soaking procedure. Because the tobacco stem raw material is a dry material, the fermentation broth can be absorbed in a large quantity and can easily penetrate into the tobacco stem, which is conducive to the uniform degradation of cell wall substances in the tobacco stem.

The tobacco stem raw material has a moisture content of 11% to 13%. The sieving is conducted with a sieve pore size of 8 mm to 10 mm for removing coarse stems and a sieve pore size of 3 mm to 4 mm for removing fine stems, which ensures the relatively uniform and consistent sizes of tobacco stems and improves the uniformity of the subsequent fermentation broth absorption, expansion, and drying. As a result, a tobacco stem obtained after the pretreatment has a diameter of 8 mm to 10 mm or 3 mm to 4 mm.

After the washing and soaking, the washed and soaked tobacco stem is delivered by a mesh belt to a fermentation bed, and free water on a surface of the washed and soaked tobacco stem is drained during the delivering. A sealing cover plate is provided above the mesh belt to prevent the excessive temperature loss and reduce the energy consumption. The solid-state fermentation is conducted for 6 h to 24 h on the fermentation bed, and the fermentation bed has a temperature of 40° C. to 60° C. and a relative humidity of 70% to 90%.

In the step 5, the drying is as follows: delivering the fermented tobacco stem to a microwave drying device, and allowing expansion, drying, and inactivation to obtain a dried fermented tobacco stem with a moisture content of 8% to 10%, where an expansion time is 30 s to 100 s and an expansion degree is 1.5 to 2.0. In the microwave drying device, under the action of microwaves, water in the fermented tobacco stem evaporates sharply to make the fermented tobacco stem expand rapidly, and during an expansion and drying process of the tobacco stem, the proteases in the fermentation broth are inactivated under the action of a high temperature. Compared with the hot-air convection drying, when the microwave drying is adopted, microwaves can directly penetrate into a tobacco stem, and the moisture inside and outside the tobacco stem is evenly distributed without a wet core phenomenon, which can effectively prevent the risk of mildew during late storage and allows a specified expansion effect for the tobacco stem.

After being dried, the fermented tobacco stem is delivered to a quantitative packing machine for packing. The dried fermented tobacco stem is delivered by a mesh belt with air blown below to cool the dried fermented tobacco stem to 35° C. or lower, and the dried fermented tobacco stem is weighed and then packed. After being packed, the dried fermented tobacco stem can be delivered to a cigarette factory for the subsequent production and processing of a cut rolled stem, such as heating and humidifying, stem storage, shredding, and fragrance addition.

The proportion “%” with regard to an amount of a substance involved in the present disclosure refers to a mass percentage, unless otherwise specified.

In the second solution, the tobacco stem washing and the fermentation broth application are completed simultaneously during the stem washing and soaking procedure, the tobacco stem fermentation and the tobacco stem storage are completed simultaneously during the tobacco stem fermentation procedure, and the tobacco stem expansion, drying, and inactivation are completed simultaneously during the expansion and drying procedure. Throughout the above process, an additional tobacco stem storage cabinet is not required, and a process flow is concise and efficient. The present disclosure has the following beneficial effects: In the present disclosure, a clear fermentation broth is prepared by cultivating Ganoderma lucidum and filtering a resulting fungal solution, and then applied to a surface of a tobacco stem or a cut rolled stem for fermentation. After the fermentation, cell wall substances such as lignin and cellulose are degraded and utilized, which reduces the occurrence of undesirable smells such as a woody smell and a pungent odor, and improves a sensory quality of the tobacco stem or the cut rolled stem.

### BRIEF DESCRIPTION OF THE DRAWINGS

FIG. 1 is a process flow chart of Examples 1 to 6 of the present disclosure; and

FIG. 2 is a process flow chart of Examples 7 to 10 of the present disclosure.

### DETAILED DESCRIPTION OF THE EMBODIMENTS

To make the objective, technical solutions, and advantages of the present disclosure comprehensible, the present disclosure will be further described below in detail below with reference to examples. It should be understood that the specific examples described herein are merely intended to explain the present disclosure, rather than to limit the present disclosure.

### 1. Addition of a Ganoderma lucidum Fermentation Broth to a Cut Rolled Stem

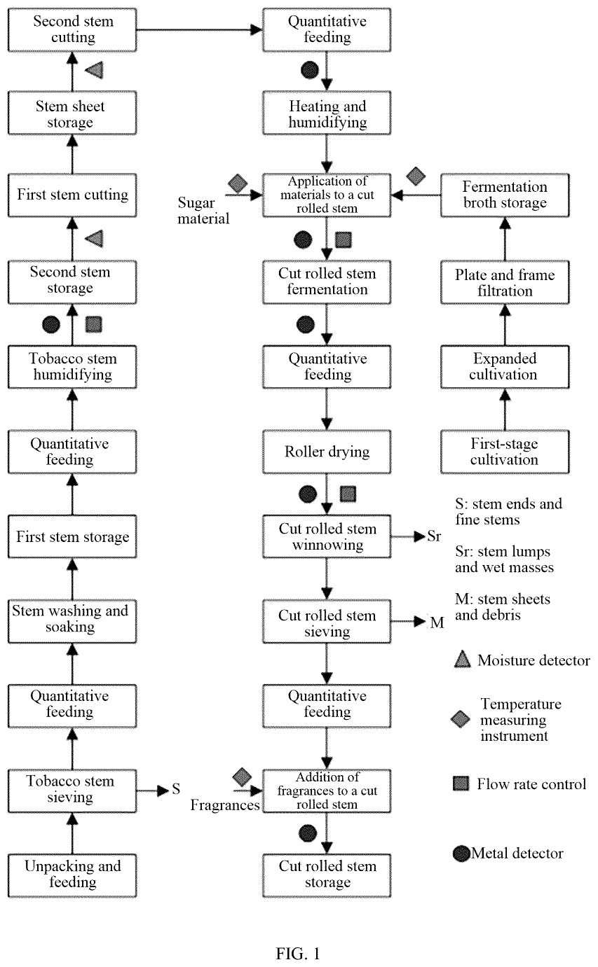

As shown in FIG. 1, it includes the following steps: tobacco stem sieving to remove stem ends and fine stems, stem washing and soaking, first stem storage, tobacco stem humidifying, second stem storage, cutting tobacco stems into stem sheets, stem sheet storage, stem sheet re-cutting, heating and humidifying, fermentation broth application, cut rolled stem fermentation, roller drying, winnowing to remove wet masses and stem lumps, re-sieving to remove stem sheets and debris, fragrance addition, and cut rolled stem storage. In the step of applying the sugar material to the cut rolled stem with the feeding machine, a fermentation broth prepared by cultivating Ganoderma lucidum is added to the cut rolled stem for fermentation.

### Example 1 Preparation of a Fermentation Broth

As shown in FIG. 1, Ganoderma lucidum was first subjected to first-stage cultivation in a PDB medium and then subjected second-stage expanded cultivation in a cultivation tank to obtain a fungal solution, then the fungal solution was filtered with a plate and frame filter to obtain the fermentation broth, and the fermentation broth was stored for later use. A specific method was as follows:

The PDB medium was prepared with 1,400 g of a potato, 160 g of glucose, and 60 L of distilled water, and had a natural pH.

First-stage cultivation: Ganoderma lucidum was inoculated into the PDB medium with an inoculum size of 3%, and cultivated at a temperature of 30° C. and a rotational speed of 100 rpm for 5 d.

A second-stage expanded cultivation medium was prepared with 3 kg of a yeast extract powder, 20 kg of a corn syrup, 31 kg of a bran, 20 kg of a tobacco stem powder, 1.1 kg of monopotassium phosphate, 1 kg of magnesium sulfate heptahydrate, 0.1 kg of vitamin B1, 0.0005 kg of a silicone oil defoaming agent, and 1,000 L of distilled water.

Second-stage expanded cultivation: A seed culture was inoculated with an inoculum size of 8% into the second-stage expanded cultivation medium, and cultivated for 4 d at a ventilation ratio of 0.6, a rotational speed of 100 rpm, and a temperature of 30° C. to obtain a fungal solution including extracellular enzymes such as laccase, cellulase, and pectinase. With the laccase in the fungal solution as a representative protease, an enzymatic activity of the laccase at the end of cultivation was 100,000 U/L.

The fungal solution in the cultivation tank was filtered by a plate and frame filter with a mesh diameter of 500 μm to remove solids such as bran, tobacco stem powder, and mycelium in the fungal solution to obtain a clear fermentation broth without obvious solids, and the fermentation broth was stored at a temperature of lower than or equal to 4° C. for later use.

### Example 2

(1) As shown in FIG. 1, a tobacco stem was washed and soaked, heated and humidified, stored, and double-cut to obtain a cut rolled stem with a moisture content of 30%.
(2) In the step of applying the sugar material to the cut rolled stem with the feeding machine, the cut rolled stem was heated and humidified (a steam pressure: 0.10 Mpa) to 45° C. to obtain a heated and humidified cut rolled stem, the fermentation broth prepared in Example 1 was diluted with water to a laccase activity of 10,000 U/L to obtain a diluted fermentation broth, and water, a sugar material, and the diluted fermentation broth were proportionally applied by a feeding machine evenly to the heated and humidified cut rolled stem, where a hot air temperature was 45° C., a water application proportion was 1.5%, a sugar material application proportion was 2.0%, and a fermentation broth application proportion was 0.5%; and then a resulting cut rolled stem was stored for 4 h in a leaf storage room with a temperature of 35° C. and a relative humidity of 85% to allow solid-state fermentation to obtain a fermented cut rolled stem.
(3) After the solid-state fermentation, the fermented cut rolled stem was expanded and inactivated by a high-temperature steam with a steam pressure of 0.4 Mpa and a steam flow rate of 700 kg/h, and then dried by a roller with a wall temperature of 120° C. and a hot air temperature of 110° C. to remove excess water to obtain a dried fermented cut rolled stem with a moisture content of 13.0%, the dried fermented cut rolled stem was subjected to winnowing to remove impurities such as stem lumps and wet masses and sieved to remove stem sheets and debris, and finally, fragrances were added to obtain a finished cut rolled stem with a moisture content of 12.0%.

A degradation rate of lignin in the cell wall substances in the finished cut rolled stem prepared by the above solution was detected by an acetyl bromide method, the degradation rate of lignin represented a degradation rate of cell wall substances, and the degradation rate of lignin was 15.62%. The finished cut rolled stem was subjected to smoking evaluation according to the “Tobacco in processing-Sensory evaluation methods” YC/T 415-2011, and results showed that a woody smell and a pungent odor of the finished cut rolled stem were reduced.

### Example 3

(1) A tobacco stem was washed and soaked, heated and humidified, stored, and double-cut to obtain a cut rolled stem with a moisture content of 30%.
(2) The cut rolled stem was heated and humidified (a steam pressure: 0.10 Mpa) to 45° C.; the fermentation broth prepared in Example 1 was diluted with water to a laccase activity of 25,000 U/L; the feeding machine had a hot air temperature of 45° C., a water application proportion of 1.5%, a sugar material application proportion of 2.0%, and a fermentation broth application proportion of 1.0%; and the leaf storage room had a temperature of 35° C. and a relative humidity of 85%, and a storage time was 14 h.
(3) For the expansion of the fermented cut rolled stem, a steam pressure was 0.4 Mpa and a steam flow rate was 700 kg/h; for the drying, a wall temperature was 120° C. and a hot air temperature was 110° C.; a dried fermented cut rolled stem produced after the drying had a moisture content of 13.0%; and a finished cut rolled stem produced after the fragrance addition had a moisture content of 12.0%. Others were the same as those in Example 2.

Lignin among the cell wall substances in the finished cut rolled stem prepared by the above solution, as a representative, was detected by an acetyl bromide method, and a degradation rate of lignin was 30.23%. The finished cut rolled stem was subjected to smoking evaluation according to the “Tobacco in processing-Sensory evaluation methods” YC/T 415-2011, and results showed that a woody smell and a pungent odor of the finished cut rolled stem were significantly reduced, and the cleanliness and comfort of a smoke of the finished cut rolled stem were increased.

### Example 4

(1) A tobacco stem was washed and soaked, heated and humidified, stored, and double-cut to obtain a cut rolled stem with a moisture content of 30%.
(2) Fermentation broth application stage: The cut rolled stem was heated and humidified (a steam pressure: 0.10 Mpa) to 45° C. to obtain a heated and humidified cut rolled stem, the fermentation broth prepared in Example 1 was diluted with water to a laccase activity of 50,000 U/L to obtain a diluted fermentation broth, and water, a sugar material, and the diluted fermentation broth were proportionally applied by a feeding machine evenly to the heated and humidified cut rolled stem, where a hot air temperature was 45° C., a water application proportion was 1.5%, a sugar material application proportion was 2.0%, and a fermentation broth application proportion was 2.0%; and then a resulting cut rolled stem was stored for 24 h in a leaf storage room with a temperature of 35° C. and a relative humidity of 85% to obtain a fermented cut rolled stem.
(3) For the expansion of the fermented cut rolled stem, a steam pressure was 0.4 Mpa and a steam flow rate was 700 kg/h; for the drying, a wall temperature was 120° C. and a hot air temperature was 110° C.; a dried fermented cut rolled stem produced after the drying had a moisture content of 13.0%; and a finished cut rolled stem produced after the fragrance addition had a moisture content of 12.0%. Others were the same as those in Example 2.

Lignin among the cell wall substances in the finished cut rolled stem prepared by the above solution, as a representative, was detected by an acetyl bromide method, and a degradation rate of lignin was 52.84%. The finished cut rolled stem was subjected to smoking evaluation according to the “Tobacco in processing-Sensory evaluation methods” YC/T 415-2011, and results showed that a woody smell and a pungent odor of the finished cut rolled stem were significantly reduced, the cleanliness and comfort of a smoke of the finished cut rolled stem were significantly increased, and the finished cut rolled stem had a slight inharmonious smell.

### Example 5

(1) A tobacco stem was washed and soaked, heated and humidified, stored, and double-cut to obtain a cut rolled stem with a moisture content of 30%.
(2) Fermentation broth application stage: The cut rolled stem was heated and humidified (a steam pressure: 0.10 Mpa) to 35° C. to obtain a heated and humidified cut rolled stem, the fermentation broth prepared in Example 1 was diluted with water to a laccase activity of 30000 U/L to obtain a diluted fermentation broth, and water, a sugar material, and the diluted fermentation broth were proportionally applied by a feeding machine evenly to the heated and humidified cut rolled stem, where a hot air temperature was 35° C., a water application proportion was 1.5%, a sugar material application proportion was 2.0%, and a fermentation broth application proportion was 2.0%; and then a resulting cut rolled stem was stored for 24 h in a leaf storage room with a temperature of 35° C. and a relative humidity of 85% to obtain a fermented cut rolled stem.
(3) For the expansion of the fermented cut rolled stem, a steam pressure was 0.4 Mpa and a steam flow rate was 700 kg/h; for the drying, a wall temperature was 120° C. and a hot air temperature was 110° C.; a dried fermented cut rolled stem produced after the drying had a moisture content of 13.0%; and a finished cut rolled stem produced after the fragrance addition had a moisture content of 12.0%. Others were the same as those in Example 2.

Lignin among the cell wall substances in the finished cut rolled stem prepared by the above solution, as a representative, was detected by an acetyl bromide method, and a degradation rate of lignin was 36.17%. The finished cut rolled stem was subjected to smoking evaluation according to the “Tobacco in processing-Sensory evaluation methods” YC/T 415-2011, and results showed that a woody smell and a pungent odor of the finished cut rolled stem were significantly reduced, the cleanliness and comfort of a smoke of the finished cut rolled stem were significantly increased, and the finished cut rolled stem was slightly fragrant and sweet and had a prominent harmony overall.

### Example 6

(1) A tobacco stem was washed and soaked, heated and humidified, stored, and double-cut to obtain a cut rolled stem with a moisture content of 30%.
(2) Fermentation broth application stage: The cut rolled stem was heated and humidified (a steam pressure: 0.10 Mpa) to 35° C. to obtain a heated and humidified cut rolled stem, the fermentation broth prepared in Example 1 was diluted with water to a laccase activity of 10,000 U/L to obtain a diluted fermentation broth, and water, a sugar material, and the diluted fermentation broth were proportionally applied by a feeding machine evenly to the heated and humidified cut rolled stem, where a hot air temperature was 35° C., a water application proportion was 1.5%, a sugar material application proportion was 2.0%, and a fermentation broth application proportion was 1.0%; and then a resulting cut rolled stem was stored for 14 h in a leaf storage room with a temperature of 35° C. and a relative humidity of 85% to obtain a fermented cut rolled stem.
(3) For the expansion of the fermented cut rolled stem, a steam pressure was 0.4 Mpa and a steam flow rate was 700 kg/h; for the drying, a wall temperature was 120° C. and a hot air temperature was 110° C.; a dried fermented cut rolled stem produced after the drying had a moisture content of 13.0%; and a finished cut rolled stem produced after the fragrance addition had a moisture content of 12.0%. Others were the same as those in Example 2.

Lignin among the cell wall substances in the finished cut rolled stem prepared by the above solution, as a representative, was detected by an acetyl bromide method, and a degradation rate of lignin was 18.54%.

TABLE 1
Conditions and degradation rates of Examples 2 to 6

| Example | Laccase | Application | Storage | Degradation |
|---|---|---|---|---|
| No. | activity, U/L | proportion, % | time, h | rate, % |
| Example 2 | 10000 | 0.5 | 4 | 15.62 |
| Example 3 | 25000 | 1.0 | 14 | 30.23 |
| Example 4 | 50000 | 2.0 | 24 | 52.84 |
| Example 5 | 30000 | 2.0 | 24 | 36.17 |
| Example 6 | 10000 | 1.0 | 14 | 18.54 |

It can be seen from Table 1 that the enzymatic activity, the application proportion, and the storage time all have significant impacts on the degradation rate, and there is the highest degradation rate of lignin in Example 4.

### 2. Addition of a Ganoderma lucidum Fermentation Broth to a Tobacco Stem

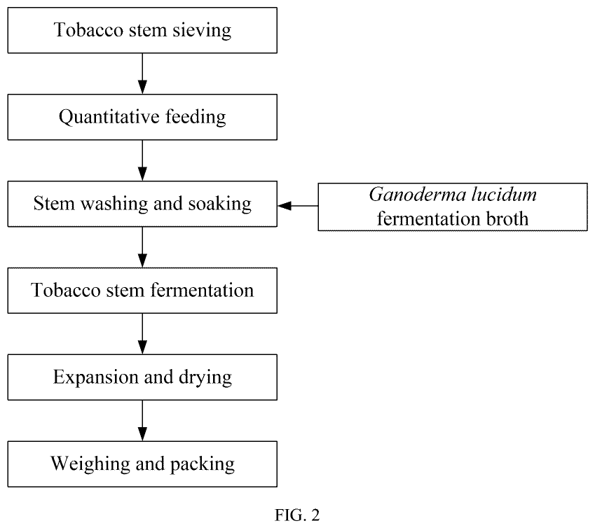

As shown in FIG. 2, a Ganoderma lucidum fermentation broth is prepared, tobacco stems are sieved, corresponding materials are quantitatively fed into a stem washing and soaking device, and the Ganoderma lucidum fermentation broth is added to the stem washing and soaking device, such that sieved tobacco stems absorb the fermentation broth during a washing and soaking process; and resulting tobacco stems are subjected to solid-state fermentation and then expansion and drying, and finally weighed and packed.

### Preparation of the Fermentation Broth and Preparation of the Tobacco Stem Raw Material

Fermentation broth preparation: Ganoderma lucidum is first cultivated on a shaker and then subjected to expanded cultivation in a fermentation tank to obtain a fungal solution including extracellular enzymes such as laccase, cellulase, and pectinase, and the fungal solution is filtered by a plate and frame filter with a mesh diameter of 500 μm to 1,000 μm to obtain a clear fermentation broth without obvious solids.

Tobacco stem sieving: A tobacco stem raw material with a moisture content of 12% is sieved with a sieve pore size of 10 mm for removing coarse stems and a sieve pore size of 4 mm for removing fine stems to remove coarse stems and fine stems.

The above fermentation broth and tobacco stem are used in Examples 7 to 10 and Comparative Examples 1 and 2.

### Example 7

(1) The fermentation broth was added to circulating water for stem washing and soaking to make the circulating water have a laccase activity of 3,000 U/L; the circulating water had a temperature of 40° C.; the stem washing and soaking was conducted for 30 s; and a tobacco stem produced after the stem washing and soaking had a moisture content of 20%.
(2) A fermentation bed had a temperature of 40° C. and a relative humidity of 70%, and a fermentation time was 6 h.
(3) An expansion and drying time was 35 s; and a tobacco stem produced after expansion and drying had a moisture content of 8% and an expansion degree of 1.5%.

Lignin among the cell wall substances in a tobacco stem prepared by the above solution, as a representative, was detected by an acetyl bromide method, and degradation rates of lignin in a skin and a core of the tobacco stem were 14.38% and 10.47%, respectively. The tobacco stem was prepared into a cut rolled stem by a conventional process, and then subjected to smoking evaluation according to the “Tobacco in processing-Sensory evaluation methods” YC/T 415-2011, and results showed that a woody smell, burning sensation, and a pungent odor of the cut rolled stem were reduced slightly.

### Example 8

(1) The fermentation broth was added to circulating water for stem washing and soaking with a laccase activity of 7,000 U/L; the circulating water had a temperature of 45° C.; the stem washing and soaking was conducted for 70 s; and a tobacco stem produced after the stem washing and soaking had a moisture content of 25%.
(2) A fermentation bed had a temperature of 45° C. and a relative humidity of 76%, and a fermentation time was 16 h.
(3) An expansion and drying time was 50 s; and a tobacco stem produced after expansion and drying had a moisture content of 8% and an expansion degree of 1.8%.

Lignin among the cell wall substances in a tobacco stem prepared by the above solution, as a representative, was detected by an acetyl bromide method, and degradation rates of lignin in a skin and a core of the tobacco stem were 30.56% and 27.29%, respectively. The tobacco stem was prepared into a cut rolled stem by a conventional process, and then subjected to smoking evaluation according to the “Tobacco in processing-Sensory evaluation methods” YC/T 415-2011, and results showed that a woody smell, burning sensation, and a pungent odor of the cut rolled stem were reduced significantly, and the cleanliness and comfort of a smoke of the cut rolled stem were increased.

### Example 9

(1) The fermentation broth was added to circulating water for stem washing and soaking with a laccase activity of 10,000 U/L; the circulating water had a temperature of 50° C.; the stem washing and soaking was conducted for 100 s; and a tobacco stem produced after the stem washing and soaking had a moisture content of 30%.
(2) A fermentation bed had a temperature of 50° C. and a relative humidity of 80%, and a fermentation time was 24 h.
(3) An expansion and drying time was 50 s; an expansion degree was 2.0%; and a tobacco stem produced after expansion and drying had a moisture content of 9.5%.

Lignin among the cell wall substances in a tobacco stem prepared by the above solution, as a representative, was detected by an acetyl bromide method, and degradation rates of lignin in a skin and a core of the tobacco stem were 49.38% and 47.96%, respectively. The tobacco stem was prepared into a cut rolled stem by a conventional process, and then subjected to smoking evaluation according to the “Tobacco in processing-Sensory evaluation methods” YC/T 415-2011, and results showed that a woody smell, burning sensation, and a pungent odor of the cut rolled stem were reduced significantly, the comfort of a smoke of the cut rolled stem was significantly increased, and the cut rolled stem had a slight inharmonious smell such as a scorching smell.

### Example 10

(1) The fermentation broth was added to circulating water for stem washing and soaking with a laccase activity of 9,000 U/L; the circulating water had a temperature of 45° C.; the stem washing and soaking was conducted for 100 s; and a tobacco stem produced after the stem washing and soaking had a moisture content of 28%.
(2) A fermentation bed had a temperature of 50° C. and a relative humidity of 78%, and a fermentation time was 16 h.
(3) An expansion and drying time was 48 s; an expansion degree was 1.5%; and a tobacco stem produced after expansion and drying had a moisture content of 9.5%.

Lignin among the cell wall substances in a tobacco stem prepared by the above solution, as a representative, was detected by an acetyl bromide method, and degradation rates of lignin in a skin and a core of the tobacco stem were 37.43% and 35.87%, respectively. The tobacco stem was prepared into a cut rolled stem by a conventional process, and then subjected to smoking evaluation according to the “Tobacco in processing-Sensory evaluation methods” YC/T 415-2011, and results showed that a woody smell, burning sensation, and a pungent odor of the cut rolled stem were reduced significantly, the cleanliness and comfort of a smoke of the cut rolled stem were significantly increased, and the cut rolled stem had a prominent harmony overall.

### Comparative Example 1

In this comparative example, the tobacco stem was first washed and soaked with tap water to obtain a washed and soaked tobacco stem with a moisture content of 28%, and then the fermentation broth was applied by a feeding machine to the washed and soaked tobacco stem, where a fermentation broth application proportion was 16%, a laccase activity of the fermentation broth was 9,000 U/L, and after the fermentation broth was applied, a resulting tobacco stem had an actual moisture content of 38.7%. Others in this comparative example were the same as those in Example 10.

Lignin among the cell wall substances in a tobacco stem prepared by the above solution, as a representative, was detected by an acetyl bromide method, and degradation rates of lignin in a skin and a core of the tobacco stem were 42.15% and 22.76%, respectively. A difference between the degradation rates of lignin in the skin and the core of the tobacco stem in this comparative example is 19.39%, and a difference between the degradation rates of lignin in the skin and the core of the tobacco stem in Example 10 is merely 1.56%. The fermentation broth acts more on a surface of the tobacco stem in this comparative example than in Example 10. It can be known that, when the fermentation broth is directly applied to a dry tobacco stem material during the stem washing and soaking procedure, the fermentation broth is easy to penetrate into the tobacco stem, and a fermentation effect is uniform. In addition, compared with Example 10, the moisture content of the tobacco stem in this comparative example is increased from 28% to 38.7%. Although the fermentation broth application proportion is 16% in this comparative example, the moisture content actually increases merely by 10.7%, indicating that, when the fermentation broth is applied to a wet tobacco stem material, the fermentation broth may not be completely absorbed by the tobacco stem. The moisture content of the tobacco stem in Example 10 is increased from 12% to 28%, with an increase of 16%, indicating that, when the fermentation broth is directly applied to a dry tobacco stem material during the stem washing and soaking procedure, an absorption capacity of the fermentation broth is large.

### Comparative Example 2

In this comparative example, the tobacco stem was subjected to hot-air convection drying, where hot air was blown from bottom to top in the first three drying zones, hot air was blown from top to bottom in the middle two drying zones, and a hot air temperature was 120° C.; natural air was blown from bottom to top in the last cooling zone; a drying time was 20 min; and a tobacco stem produced after the hot-air convection drying had a moisture content of 11%. Others in this comparative example were the same as those in Example 10.

Moisture contents of tobacco stems with different sizes were detected by an oven method. In this comparative example, moisture contents in a skin and a core of a tobacco stem with a diameter of 4 mm were 9.36% and 10.68%, respectively, and moisture contents in a skin and a core of a tobacco stem with a diameter of 8 mm were 9.79% and 14.31%, respectively. In Example 10, moisture contents in a skin and a core of a tobacco stem with a diameter of 4 mm were 9.38% and 9.55%, respectively, and moisture contents in a skin and a core of a tobacco stem with a diameter of 8 mm were 9.41% and 9.57%, respectively. Compared with Example 10, in this comparative example, there is a large difference in a moisture content between the tobacco stems of different sizes and between a skin and a core of a tobacco stem. In particular, a coarse tobacco stem undergoes a wet core phenomenon and has a specified risk of mildew during late storage. In Example 10, there is a small difference in a moisture content between the tobacco stems and between a skin and a core of a tobacco stem, indicating that the microwave drying is conducive to improving the consistency of moisture contents of tobacco stems. In addition, compared with Example 10, in this comparative example, water is heated with a natural gas to produce a steam, then air is heated with the steam to produce hot air, and finally a tobacco stem is dried with the hot air, where a drying time is as long as 25 min. In Example 10, microwaves are produced directly with electric energy to dry a tobacco stem, where a drying time is merely 48 s. It can be seen that the microwave drying can reduce an energy conversion process and improve an energy utilization efficiency and a production and processing efficiency.

Although the present disclosure is described herein with reference to the interpretative embodiments of the present disclosure, it should be understood that a person skilled in the art may design many other modifications and embodiments, and these modifications and embodiments will fall within the scope and spirit of the principles of the present disclosure. Specifically, within the scope of the disclosure of the present disclosure, a plurality of variations and improvements may be made to constituent components and/or a layout of the subject matter. In addition to the variations and improvements made to the constituent parts and/or the layout, other alterations will be apparent to those skilled in the art.

What is claimed is:

1. A method for improving a sensory quality of a cut rolled stem using a Ganoderma lucidum fermentation broth, comprising the following steps:
step 1, a fermentation broth preparation: subjecting Ganoderma lucidum to a first-stage cultivation and then to a second-stage expanded cultivation in a fermentation tank to obtain a fungal solution, and filtering the fungal solution to obtain a clear fermentation broth;
step 2, conducting a pretreatment on a tobacco stem to obtain a pretreated tobacco stem or the cut rolled stem, wherein the pretreatment is one or more treatments of a sieving, a stem washing and soaking, a heating and humidifying, a stem storage, and a shredding;
step 3, a fermentation broth application: applying the clear fermentation broth to the pretreated tobacco stem or the cut rolled stem;
step 4, a solid-state fermentation: subjecting an applied tobacco stem or an applied cut rolled stem to the solid-state fermentation to obtain a fermented tobacco stem or a fermented cut rolled stem; and
step 5, drying the fermented tobacco stem or the fermented cut rolled stem.

2. The method for improving the sensory quality of the cut rolled stem using the Ganoderma lucidum fermentation broth according to claim 1, wherein in the step 1, a medium for the first-stage cultivation and the second-stage expanded cultivation is a potato dextrose broth (PDB) medium or a potato dextrose agar (PDA) medium; and at an end of the second-stage expanded cultivation, an enzymatic activity of laccase of the fungal solution is 50,000 U/L to 300,000 U/L.

3. The method for improving the sensory quality of the cut rolled stem using the Ganoderma lucidum fermentation broth according to claim 1, wherein in the step 1, the fungal solution is filtered with a plate and frame filter, and the plate and frame filter has a mesh diameter of smaller than or equal to 1,000 μm.

4. The method for improving the sensory quality of the cut rolled stem using the Ganoderma lucidum fermentation broth according to claim 1, wherein in the step 4, the solid-state fermentation is conducted for 4 h to 24 h at a fermentation temperature of 30° C. to 60° C. and a relative humidity of 70% to 90%.

5. The method for improving the sensory quality of the cut rolled stem using the Ganoderma lucidum fermentation broth according to claim 1, wherein in the step 2, the pretreatment comprises the sieving, the stem washing and soaking, the heating and humidifying, the stem storage, and the shredding, sequentially, and after the pretreatment, the cut rolled stem is obtained; before the clear fermentation broth is applied, the cut rolled stem is heated and humidified; and the clear fermentation broth is applied as follows: spraying a diluted clear fermentation broth by a feeding machine on a surface of a heated and humidified cut rolled stem with an application proportion of 0.5 wt % to 2.0 wt %.

6. The method for improving the sensory quality of the cut rolled stem using the Ganoderma lucidum fermentation broth according to claim 5, wherein after the clear fermentation broth is diluted, an enzymatic activity of laccase of the diluted clear fermentation broth is 10,000 U/L to 50,000 U/L.

7. The method for improving the sensory quality of the cut rolled stem using the Ganoderma lucidum fermentation broth according to claim 6, wherein the solid-state fermentation is conducted in a leaf storage room, the leaf storage room has a temperature of 30° C. to 40° C. and a relative humidity of 70% to 90%, and a storage time is 4 h to 24 h.

8. The method for improving the sensory quality of the cut rolled stem using the Ganoderma lucidum fermentation broth according to claim 7, wherein in the step 5, the fermented cut rolled stem is expanded and inactivated with a steam before being dried, and then dried with a roller to obtain a dried fermented cut rolled stem with a moisture content of 12.5% to 13.5%.

9. The method for improving the sensory quality of the cut rolled stem using the Ganoderma lucidum fermentation broth according to claim 7, wherein for an expansion of the fermented cut rolled stem, a steam pressure is 0.3 MPa to 0.5 MPa and a steam flow rate is 600 kg/h to 900 kg/h.

10. The method for improving the sensory quality of the cut rolled stem using the Ganoderma lucidum fermentation broth according to claim 1, wherein in the step 2, the pretreatment comprises the sieving, and after the pretreatment, the pretreated tobacco stem is obtained; and the clear fermentation broth is applied as follows: adding the clear fermentation broth to circulating water in a stem washing and soaking device, and with an enzymatic activity of laccase in the circulating water in the stem washing and soaking device controlled at 3,000 U/L to 10,000 U/L and a temperature of the circulating water controlled at 40° C. to 60° C., washing and soaking the pretreated tobacco stem for 20 s to 120 s to obtain a washed and soaked tobacco stem with a moisture content of 20% to 30%, wherein after the pretreated tobacco stem is washed and soaked, the clear fermentation broth is absorbed, and impurities are washed away.

11. The method for improving the sensory quality of the cut rolled stem using the Ganoderma lucidum fermentation broth according to claim 10, wherein the pretreated tobacco stem has a diameter of 8 mm to 10 mm or 3 mm to 4 mm; and a tobacco stem raw material has a moisture content of 11% to 13%.

12. The method for improving the sensory quality of the cut rolled stem using the Ganoderma lucidum fermentation broth according to claim 10, wherein after the pretreated tobacco stem is washed and soaked, the washed and soaked tobacco stem is delivered by a mesh belt to a fermentation bed, and free water on a surface of the washed and soaked tobacco stem is drained during a delivering.

13. The method for improving the sensory quality of the cut rolled stem using the Ganoderma lucidum fermentation broth according to claim 12, wherein the solid-state fermentation is conducted for 6 h to 24 h on the fermentation bed, and the fermentation bed has a temperature of 40° C. to 60° C. and a relative humidity of 70% to 90%.

14. The method for improving the sensory quality of the cut rolled stem using the Ganoderma lucidum fermentation broth according to claim 10, wherein in the step 5, the fermented tobacco stem is dried as follows: delivering the fermented tobacco stem to a microwave drying device, and allowing an expansion, a drying, and an inactivation to obtain a dried fermented tobacco stem with a moisture content of 8% to 10% and an expansion degree of 1.5 to 2.0, wherein an expansion time is 30 s to 100 s.

15. The method for improving the sensory quality of the cut rolled stem using the Ganoderma lucidum fermentation broth according to claim 2, wherein in the step 2, the pretreatment comprises the sieving, the stem washing and soaking, the heating and humidifying, the stem storage, and the shredding, sequentially, and after the pretreatment, the cut rolled stem is obtained; before the clear fermentation broth is applied, the cut rolled stem is heated and humidified; and the clear fermentation broth is applied as follows: spraying a diluted clear fermentation broth by a feeding machine on a surface of a heated and humidified cut rolled stem with an application proportion of 0.5 wt % to 2.0 wt %.

16. The method for improving the sensory quality of the cut rolled stem using the Ganoderma lucidum fermentation broth according to claim 3, wherein in the step 2, the pretreatment comprises the sieving, the stem washing and soaking, the heating and humidifying, the stem storage, and the shredding, sequentially, and after the pretreatment, the cut rolled stem is obtained; before the clear fermentation broth is applied, the cut rolled stem is heated and humidified; and the clear fermentation broth is applied as follows: spraying a diluted clear fermentation broth by a feeding machine on a surface of a heated and humidified cut rolled stem with an application proportion of 0.5 wt % to 2.0 wt %.

17. The method for improving the sensory quality of the cut rolled stem using the Ganoderma lucidum fermentation broth according to claim 4, wherein in the step 2, the pretreatment comprises the sieving, the stem washing and soaking, the heating and humidifying, the stem storage, and the shredding, sequentially, and after the pretreatment, the cut rolled stem is obtained; before the clear fermentation broth is applied, the cut rolled stem is heated and humidified; and the clear fermentation broth is applied as follows: spraying a diluted clear fermentation broth by a feeding machine on a surface of a heated and humidified cut rolled stem with an application proportion of 0.5 wt % to 2.0 wt %.

18. The method for improving the sensory quality of the cut rolled stem using the Ganoderma lucidum fermentation broth according to claim 15, wherein after the clear fermentation broth is diluted, an enzymatic activity of laccase of the diluted clear fermentation broth is 10,000 U/L to 50,000 U/L.

19. The method for improving the sensory quality of the cut rolled stem using the Ganoderma lucidum fermentation broth according to claim 16, wherein after the clear fermentation broth is diluted, an enzymatic activity of laccase of the diluted clear fermentation broth is 10,000 U/L to 50,000 U/L.

20. The method for improving the sensory quality of the cut rolled stem using the Ganoderma lucidum fermentation broth according to claim 17, wherein after the clear fermentation broth is diluted, an enzymatic activity of laccase of the diluted clear fermentation broth is 10,000 U/L to 50,000 U/L.
